(19) United States
(12) Patent Application Publication
(10) Pub. No.: US 2024/0299703 A1
(43) Pub. Date: Sep. 12, 2024

(54) CATHETER

(71) Applicant: TERUMO KABUSHIKI KAISHA, Tokyo (JP)

(72) Inventors: Yuya OTAKE, Shizuoka (JP); Tetsuya FUKUOKA, Kanagawa (JP)

(73) Assignee: TERUMO KABUSHIKI KAISHA, Tokyo (JP)

(21) Appl. No.: 18/586,957

(22) Filed: Feb. 26, 2024

(30) Foreign Application Priority Data
Mar. 7, 2023 (JP) 2023-034353

(51) Int. Cl. A61M 25/00

(52) U.S. Cl. CPC A61M 25/0068; A61M 25/0023

(57) ABSTRACT

A catheter includes an outward protruding portion provided at a distalmost end portion and protruding outward in a radial direction; and an inward protruding portion provided at the distalmost end portion and protruding inward in the radial direction.

### CROSS-REFERENCES TO RELATED APPLICATIONS

This application claims priority to Japanese Patent Application No. 2023-34353 filed on Mar. 7, 2023, the entire content of which is incorporated herein by reference.

### TECHNOLOGICAL FIELD

The present invention generally relates to a catheter.

### BACKGROUND DISCUSSION

A guiding catheter is one kind of catheter and is a catheter that may be used when a balloon catheter or the like for treating a coronary artery of a heart, a lower limb artery, or the like is inserted to a target site. An example is disclosed in Japanese Patent Application Publication No. 2006-288670 (JP 2006-288670 A).

### SUMMARY

In particular, in a guiding catheter used for intravascular treatment of a coronary artery, a lower limb artery, or the like, since the diameter of the blood vessel that is a treatment target is large, it is preferable that in order to select a treatment device having a large outer diameter or to deliver a plurality of treatment devices to a treatment target site at once, or in order to deliver more treatment devices to a distal end at once, the inner diameter of the guiding catheter is increased to make the thickness of the guiding catheter thin.

In such a guiding catheter that is thin, a distalmost end portion has a round shape to avoid damage to the blood vessel, but since the guiding catheter is thin, there is a possibility that when the catheter is advanced to the vicinity of the treatment target site, the catheter unintentionally scrapes, for example, tissues such as plaque present on a blood vessel wall and accumulated in an abdominal aorta.

The catheter disclosed here is capable of suppressing scraping of tissues such as plaque present on a blood vessel wall in the catheter that is thin.

The disclosed catheter that is configured to achieve the result described above includes the following. A catheter having a hollow shape, including: an outward protruding portion provided at a distalmost end portion and protruding outward in a radial direction; and an inward protruding portion provided at the distalmost end portion and protruding inward in the radial direction.
(2) The catheter according to (1) further including a distal end tip, in which the outward protruding portion and the inward protruding portion are provided at the distal end tip.
(3) The catheter according to (1) or (2), in which a distal end portion of the outward protruding portion has a round shape.
(4) The catheter according to any one of (1) to (3), in which a distal end portion of the inward protruding portion has a round shape.
(5) The catheter according to any one of (1) to (4), wherein the catheter is configured to satisfy the following relational expressions

### D1>D2,

d1<d2, and

### T1>T2

wherein D1 is a maximum outer diameter of the distalmost end portion, d1 is a minimum inner diameter of the distalmost end portion, T1 is a maximum thickness of the distalmost end portion, D2 is an outer diameter of a proximal portion provided on a proximal end side with respect to the distalmost end portion, d2 is an inner diameter of the proximal portion, and T2 is a thickness of the proximal portion.

According to the catheter configured as described above, only the distalmost end portion can be made thick while the proximal portion provided on the proximal end side with respect to the distalmost end portion is made thin. Therefore, it is possible to provide the guiding catheter capable of suppressing scraping of tissues such as plaque present on a blood vessel wall in the guiding catheter that is thin.

### BRIEF DESCRIPTION OF THE DRAWINGS

FIG. 1 is an overall view illustrating a catheter according to an embodiment of the present invention;

FIG. 2 is a sectional front view illustrating the catheter according to the present embodiment;

FIG. 3 is a sectional front view illustrating the vicinity of a distal end of the catheter according to the present embodiment; and

FIG. 4 is a sectional front view illustrating a catheter according to a modification example.

### DETAILED DESCRIPTION

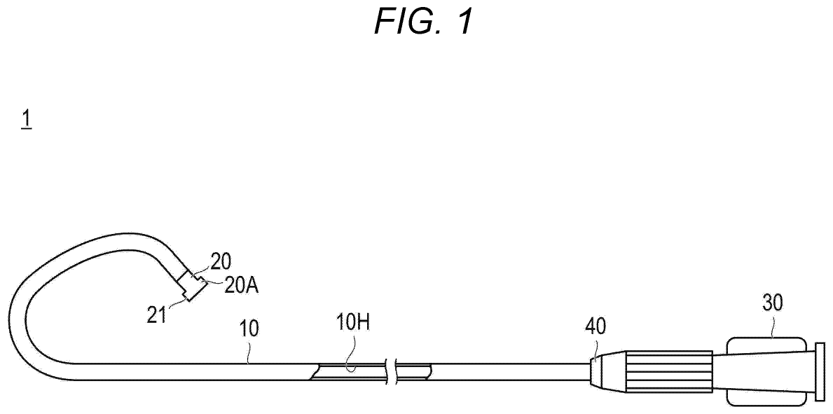

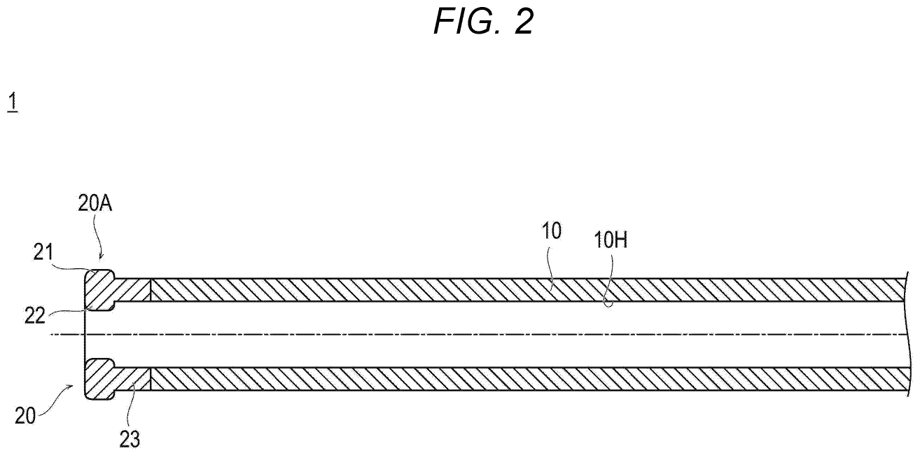

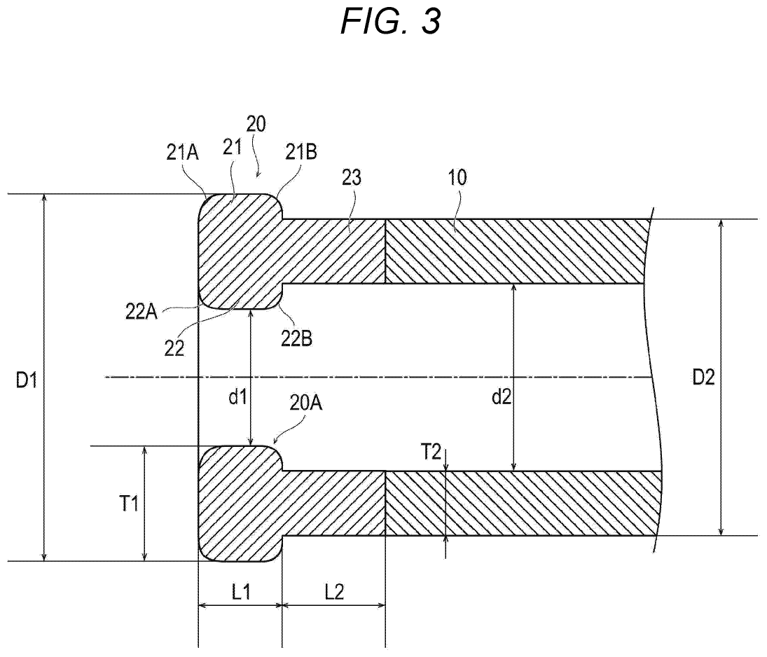

Hereinafter, a catheter 1 according to a disclosed embodiment, representing one example of the new catheter disclosed here, will be described with reference to FIGS. 1 to 3. FIG. 1 is an overall view illustrating the catheter 1 according to the embodiment. FIG. 2 is a sectional front view illustrating the catheter 1 according to the present embodiment. FIG. 3 is a sectional front view illustrating the vicinity of a distal end of the catheter 1 according to the present embodiment.

The catheter 1 illustrated in FIG. 1 is used as, for example, a guiding catheter that guides a treatment catheter (device), such as a dilation catheter (balloon catheter) for PTCA or a catheter (catheter for transporting a stent) for conveying a stent in a reduced diameter state to a stenosed site, increasing the diameter at the stenosed site, and indwelling the stent to widen and maintain the stenosed site, to a target site such as a stenosed site of a coronary artery or a lower limb artery.

As illustrated in FIGS. 1 to 3, the catheter 1 is an elongated tube-shaped member and includes a tube 10; a distal end tip 20 that is flexible and that is provided on a distal end side of the tube 10; a hub 30 provided on a proximal end side of the tube 10; and a strain relief 40 provided on the distal end side of the hub 30.

The tube 10 is formed of a tubular body having flexibility. A lumen 10H is formed in the tube 10 over the entire length of the tube 10. The lumen 10H is open at a distal end of the distal end tip 20.

The tube 10 includes an inner layer disposed on an inner surface side; an outer layer disposed around an outer periphery of the inner layer; and a reinforcing material layer disposed inside the outer layer.

It is preferable that the outer layer is more flexible on the distal end side than on the proximal end side. With such a configuration, the tube 10 can gradually increase in flexibility in a distal end direction, and can be more safely inserted into a blood vessel while ensuring sufficient pushability and torque transmission to the distal end side during an operation of inserting the tube 10 into the blood vessel.

Examples of the material constituting the outer layer include various thermoplastic elastomers such as styrene, polyolefin, polyurethane, polyester, polyamide, polybutadiene, trans-polyisoprene, fluororubber, and chlorinated polyethylene-based thermoplastic elastomers. One or a combination of two or more of these materials (polymer alloy, polymer blend, laminate, or the like) can be used.

It is preferable that the constituent material from which the inner layer is fabricated is a material that allows at least a portion in contact with a device such as a treatment catheter or a guide wire to have low friction when the device is inserted into the lumen 10H. According to this configuration, the device inserted into the tube 10 is movable in a longitudinal direction with smaller sliding resistance, and operability can be improved. Specifically, examples of the constituent material from which the inner layer may be fabricated include a fluorine-based resin material such as polytetrafluoroethylene (PTFE).

The reinforcing material layer includes a plurality of reinforcing wires that reinforce the tube 10. Examples of the reinforcing wire include a spiral or net-shaped reinforcing wire. The reinforcing wire is made of metal such as stainless steel. As specific examples, reinforcing wires are formed by crushing stainless steel wires into a flat plate shape so as to make the thickness of the tube 10 thin in a radial direction, and by spiraling or weaving (braided body) a plurality of approximately 8 to 32 stainless steel wires. It is preferable that the number of the reinforcing wires is set to a multiple of 8 to implement reinforcement in a tubular shape in a well-balanced manner.

Since the reinforcing wires uniformly receive external stress by being formed into a flat plate compared to an ellipse, physical properties become constant.

It should be noted that the number of the layers constituting the tube 10 or the constituent material of each layer may be different along the longitudinal direction of the tube 10. For example, in order to make a portion on the distal end side of the tube 10 more flexible, the number of the layers can be reduced, a more flexible material can be used, or the disposition of a reinforcing material can be omitted only at the portion.

Since the insertion of the catheter 1 into a body is performed while confirming the position thereof under X-ray fluoroscopy, it is preferable that a radiopaque material (X-ray contrast agent) is blended with the constituent material of an outer layer 12. As the radiopaque material, for example, barium sulfate, bismuth oxide, tungsten, or the like can be used. Furthermore, the proportion of blending of the radiopaque material with the constituent material of the outer layer 12 is preferably 30 to 80 wt %.

In addition, such a radiopaque material is not limited to being present over the entire length of the tube 10, and may be present in a part of the tube 10, for example, in only a distal end portion or only the distal end tip 20.

The distal end portion of the tube 10 is curved in a desired shape suitable for a site into which the distal end portion of the tube 10 is inserted, such as a left coronary artery and a right coronary artery. In particular, the distal end portion has a shape that facilitates the operation of engaging with a coronary ostium (engagement operation), or a shape that can more reliably maintain a state of engagement with the coronary ostium (engagement), but may have a linear shape without the noted distal end shape when used for lower limb artery or the like.

The distal end tip 20 is connected to a distal end of the tube 10. The distal end tip 20 is made of a material having high flexibility. By providing the distal end tip 20, the tube 10 is allowed to smoothly and safely travel in a curved, bent, or branched blood vessel.

As illustrated in FIGS. 1 to 3, the distal end tip 20 includes an outward protruding portion 21 provided at a distalmost end portion 20A and protruding outward in the radial direction; an inward protruding portion 22 provided at the distalmost end portion 20A and protruding inward in the radial direction; and an extension portion 23 provided on the proximal end side of the distalmost end portion 20A and extending in an axial direction. The outward protruding portion 21 and the inward protruding portion 22 are disposed to have the same length along the axial direction. In the present specification, an axial region in which the outward protruding portion 21 and the inward protruding portion 22 are provided is referred to as the “distalmost end portion 20A”. The distal end tip 20 is provided with a lumen communicating with the lumen 10H of the tube 10.

A distal end portion 21A of the outward protruding portion 21 has a round (rounded) shape. According to this configuration, since the distal end portion 21A has a round shape, compared to a configuration not having a round shape, it is possible to suppress scraping of tissues such as plaque present on a blood vessel wall when the catheter 1 is moved to the distal end side (in the distal or forward direction).

A proximal end portion 21B of the outward protruding portion 21 has a round (rounded) shape. According to this configuration, since the proximal end portion 21B has a round shape, compared to a configuration not having a round shape, it is possible to suppress scraping of tissues such as plaque present on a blood vessel wall when the catheter 1 is moved to the proximal end side (in the proximal or rearward direction).

A distal end portion 22A of the inward protruding portion 22 has a round (rounded) shape. According to this configuration, since the distal end portion 22A has a round shape, compared to a configuration not having a round shape, it is possible to suppress scraping of tissues such as plaque present on a blood vessel wall when the catheter 1 is moved to the distal end side (in the distal or forward direction). Thus, as shown in FIG. 3, the outer peripheral surface of the outward protruding portion 21 intersects the axially facing distal end surface (left facing end surface in FIG. 3) at rounded corners 21A and 22A.

A proximal end portion 22B of the inward protruding portion 22 has a round (rounded) shape. According to this configuration, since the proximal end portion 22B has a round shape, compared to a configuration not having a round shape, it is possible to suppress scraping of tissues such as plaque present on a blood vessel wall when the catheter 1 is moved to the proximal end side (in the proximal or rearward direction).

As described above, since the distal end tip 20 includes the outward protruding portion 21 and the inward protruding portion 22, it is possible to suppress scraping of tissues such as plaque present on a blood vessel wall when the catheter 1 is moved.

The configuration including the outward protruding portion 21 and the inward protruding portion 22 described above can be rephrased or described in a different way as follows.

As illustrated in FIG. 3, when a maximum outer diameter of the distalmost end portion 20A is D1, a minimum inner diameter of the distalmost end portion 20A is d1, a maximum thickness of the distalmost end portion 20A is T1, an outer diameter of the tube 10 (corresponding to a proximal portion) provided on the proximal end side with respect to the distalmost end portion 20A is D2, an inner diameter of the tube 10 is d2, and a thickness of the tube 10 is T2, the dimensional relationship can be expressed by a relational expression of D1>D2, d1<d2, and T1>T2.

The maximum outer diameter D1 of the distalmost end portion 20A is preferably 1.4 to 3.1 mm, the minimum inner diameter d1 of the distalmost end portion 20A is preferably 1.2 to 3 mm, and the maximum thickness T1 of the distalmost end portion 20A is preferably 0.09 to 0.4 mm.

In addition, the outer diameter D2 of the tube 10 is preferably 1.35 mm or more and 3 mm or less. In a case where the outer diameter D2 is too large, the operability of the tube 10 deteriorates when inserted and moved in an artery, and the burden on a patient increases, which is a risk.

In addition, the inner diameter d2 of the tube 10 is preferably 1.2 mm or more and 2.85 mm or less. In a case where the inner diameter d2 is too small, a treatment catheter or the like that can be inserted into the tube 10 is also reduced in outer diameter accordingly, and the range of selection of devices that may be inserted and used is limited, which is not preferable.

In addition, the thickness T2 of the tube 10 is preferably 0.05 to 0.3 mm.

In the tube 10, d2/D2 that is the ratio of the inner diameter to the outer diameter is 0.85 or more and 0.95 or less, and preferably 0.87 or more and 0.93 or less. In a case where the value of d2/D2 is too small, the result is that the thickness of the tube 10 becomes thick and the inner diameter becomes small, whereby devices that can be introduced into the tube 10 are limited. In addition, in a case where the value of d2/D2 is too large, the thickness of the tube 10 cannot be sufficiently obtained (the thickness of the tube is insufficient), so that the backup force becomes weak and kink resistance during use decreases.

In addition, the amount of outward protrusion of the outward protruding portion 21 with respect to the extension portion 23 is preferably more than 0 to 0.3 mm, and more preferably 0.05 to 0.1 mm. The amount of inward protrusion of the inward protruding portion 22 with respect to the extension portion 23 is preferably more than 0 to 0.3 mm, and more preferably 0.05 to 0.1 mm.

The extension portion 23 is provided on the proximal end side of the distalmost end portion 20A. An outer diameter of the extension portion 23 is substantially the same as the outer diameter D2 of the tube 10, and an inner diameter of the extension portion 23 is substantially the same as the inner diameter d2 of the tube 10.

A length L1 of the distalmost end portion 20A along the axial direction is preferably more than 0 to 1 mm, and more preferably 0.05 to 0.3 mm. A length L2 of the extension portion 23 along the axial direction is preferably more than 0 to 5 mm, and more preferably 0.5 to 1 mm.

It should be noted that since only the distalmost end portion 20A is thick and the proximal end side of the distalmost end portion 20A is thin in the catheter 1 described above, it is easy to cause a large treatment device or a plurality of treatment devices to protrude from the inside of the catheter 1.

Examples of the material constituting the distal end tip 20 include various rubber materials such as natural rubber, isoprene rubber, butadiene rubber, chloroprene rubber, silicone rubber, fluororubber, and styrene-butadiene rubber or various thermoplastic elastomers such as styrene, polyolefin, polyurethane, polyester, polyamide, polybutadiene, trans-polyisoprene, fluororubber, and chlorinated polyethylene-based thermoplastic elastomers.

In addition, the radiopaque material (X-ray contrast agent) as described above may be blended with the constituent material of the distal end tip 20.

The hub 30 is mounted (fixed) on a proximal end of the tube 10. A lumen communicating with the lumen 10H is formed in the hub 30. This lumen has an inner diameter substantially equal to an inner diameter of the lumen 10H, and is continuous with an inner surface of a proximal end portion of the lumen 10H without creating a step or the like.

For example, an elongated object (wire-shaped body) such as a guide wire, a catheter (for example, a balloon catheter for PTCA or a catheter for conveying a stent), an endoscope, an ultrasonic probe, or a temperature sensor can be inserted or removed from the hub 30, or various liquids such as a contrast agent (X-ray contrast agent), a drug solution, and a physiological salt solution can be injected into the hub 30. In addition, for example, the hub 30 can also be connected to other instruments such as a Y-shaped branch connector.

As described above, the catheter 1 according to the present embodiment is the catheter 1 having a hollow shape, and includes the outward protruding portion 21 provided at the distalmost end portion 20A and protruding outward in the radial direction and the inward protruding portion 22 provided at the distalmost end portion 20A and protruding inward in the radial direction. According to the catheter 1 configured in this manner, only the distalmost end portion 20A can be made thick (relatively thick) while the proximal portion (corresponding to the tube 10 or the extension portion 23) provided on the proximal end side with respect to the distalmost end portion 20A is made thin (relatively thin). Therefore, it is possible to provide the catheter 1 capable of suppressing scraping of tissues such as plaque present on a blood vessel wall in the catheter 1 that is thin.

In addition, the catheter 1 includes the distal end tip 20, and the outward protruding portion 21 and the inward protruding portion 22 are provided at the distal end tip 20. According to the catheter 1 configured in this manner, since the outward protruding portion 21 and the inward protruding portion 22 are provided at the distal end tip 20 that is more flexible, it is possible to more suitably suppress scraping of tissues such as plaque present on a blood vessel wall.

In addition, the distal end portion 21A of the outward protruding portion 21 and the distal end portion 22A of the inward protruding portion 22 have a round (rounded) shape. According to the catheter 1 configured in this manner, it is possible to more suitably suppress scraping of tissues such as plaque present on a blood vessel wall.

In addition, when the maximum outer diameter of the distalmost end portion 20A is D1, the minimum inner diameter of the distalmost end portion 20A is d1, the maximum thickness of the distalmost end portion 20A is T1, the outer diameter of the tube 10 (corresponding to the proximal portion) provided on the proximal end side with respect to the distalmost end portion 20A is D2, the inner diameter of the tube 10 is d2, and the thickness of the tube 10 is T2, the dimensional relationship is expressed by a relational expression of D1>D2, d1<d2, and T1>T2. According to the catheter 1 configured in this manner, only the distalmost end portion 20A can be made thick (relatively thick) while the proximal portion (corresponding to the tube 10 or the extension portion 23) provided on the proximal end side with respect to the distalmost end portion 20A is made thin (relatively thin). Therefore, it is possible to provide the catheter 1 capable of suppressing scraping of tissues such as plaque present on a blood vessel wall in the catheter 1 that is thin.

The catheter 1 disclosed here has been described above through the embodiment; however, the present invention is not limited to only the configurations described in the embodiment, and can be changed as appropriate within the scope of the claims.

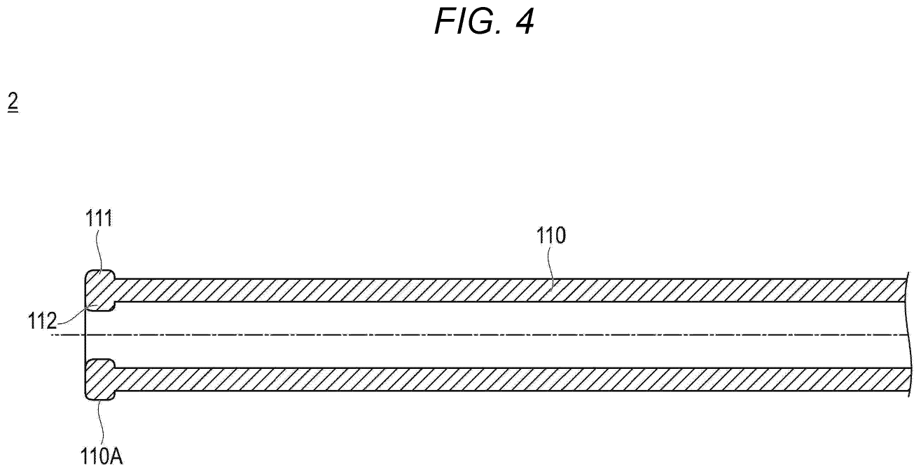

For example, in the above-described embodiment, the outward protruding portion 21 and the inward protruding portion 22 are provided at the distal end tip 20. However, as illustrated in FIG. 4, a catheter 2 according to a modification example may have a configuration in which the distal end tip 20 is not provided and an outward protruding portion 111 and an inward protruding portion 112 are provided at a distalmost end portion 110A of a tube 110.

In addition, in the above-described embodiment, the distal end portion 21A of the outward protruding portion 21, the proximal end portion 21B of the outward protruding portion 21, the distal end portion 22A of the inward protruding portion 22, and the proximal end portion 22B of the inward protruding portion 22 have a round (rounded) shape. However, the distal end portion of the outward protruding portion, the proximal end portion of the outward protruding portion, the distal end portion of the inward protruding portion, and the proximal end portion of the inward protruding portion may not have a round (rounded) shape.

The detailed description above describes embodiments of a catheter representing an example of the new catheter disclosed here. The invention is not limited, however, to the precise embodiment and variations described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims.

What is claimed is:

1. A catheter comprising:
a tube-shaped member having a distalmost end and a proximalmost end at opposite ends of the tube-shaped member, the tube-shaped member also having a lumen passing through the tube-shaped member and communicating with both a first open end of the tube-shaped member located at the distalmost end of the tube-shaped member and a second open end of the tube-shaped member at the proximalmost end of the tube-shaped member;
the tube-shaped member including a distalmost end portion, the distalmost end portion of the tube-shaped member having an axial extent extending from a distal end of the distalmost end portion to a proximal end of the distalmost end portion, the distal end of the distalmost end portion being at the distalmost end of the tube-shaped member;
the tube-shaped member including an immediately adjacent portion that is immediately proximal of the distalmost end portion;
the distalmost end portion of the tube-shaped member having an outer diameter greater than an outer diameter of the immediately adjacent portion of the tube-shaped member; and
the distalmost end portion of the tube-shaped member having an inner diameter smaller than an inner diameter of the immediately adjacent portion of the tube-shaped member.

2. The catheter according to claim 1, wherein the tube-shaped member comprises: a tube having a distal end and a proximal end; a distal end tip fixed to the distal end of the tube; and a hub fixed to the proximal end of the tube.

3. The catheter according to claim 1, wherein the tube-shaped member comprises: a tube having a distal end and a proximal end; and a distal end tip fixed to the distal end of the tube, the distalmost end portion of the tube-shaped member having the outer diameter greater than the outer diameter of the immediately adjacent portion of the tube-shaped member and having the inner diameter smaller than the inner diameter of the immediately adjacent portion of the tube-shaped member being a part of the distal end tip.

4. The catheter according to claim 1, wherein the distalmost end portion of the tube-shaped member having the outer diameter greater than the outer diameter of the immediately adjacent portion of the tube-shaped member has a radially outwardly facing outer peripheral surface and an axially facing distal end surface, the radially outwardly facing outer peripheral surface of the distalmost end portion of the tube-shaped member and the axially facing distal end surface of the distalmost end portion of the tube-shaped member intersecting one another at a rounded corner.

5. The catheter according to claim 1, wherein a distal portion of the tube-shaped member is curved.

6. The catheter according to claim 1, wherein the catheter satisfies a relational expression of
D1>D2,
d1<d2, and
T1>T2
D1 is the outer diameter of the distalmost end portion, d1 is the inner diameter of the distalmost end portion, T1 is a maximum thickness of the distalmost end portion, D2 is an outer diameter of the immediately adjacent portion of the tube-shaped member, d2 is the inner diameter of the immediately adjacent portion of the tube-shaped member, and T2 is a thickness of the immediately adjacent portion of the tube-shaped member.

7. A catheter comprising:
a tube-shaped member having a distalmost end and a proximalmost end at opposite ends of the tube-shaped member, the tube-shaped member also having a lumen passing through the tube-shaped member and communicating with both a first open end of the tube-shaped member located at the distalmost end of the tube-shaped member and a second open end of the tube-shaped member at the proximalmost end of the tube-shaped member;
the tube-shaped member including a distalmost end portion, the distalmost end portion of the tube-shaped member having an axial extent extending from a distal end of the distalmost end portion to a proximal end of the distalmost end portion, the distal end of the distalmost end portion being at the distalmost end of the tube-shaped member; and
an entirety of the axial extent of the distalmost end portion of the tube-shaped member including a protruding portion that protrudes either radially outwardly or radially inwardly relative to an immediately adjacent portion of the tube-shaped member that is immediately proximal of the tube-shaped member.

8. The catheter according to claim 7, wherein the tube-shaped member comprises: a tube having a distal end and a proximal end; a distal end tip fixed to the distal end of the tube; and a hub fixed to the proximal end of the tube.

9. The catheter according to claim 7, wherein the protruding portion protrudes radially outwardly relative to the immediately adjacent portion of the tube-shaped member.

10. The catheter according to claim 7, wherein the protruding portion protrudes radially inwardly relative to the immediately adjacent portion of the tube-shaped member.

11. The catheter according to claim 7, wherein the protruding portion protrudes radially outwardly relative to the immediately adjacent portion of the tube-shaped member, the entirety of the axial extent of the distalmost end portion of the tube-shaped member including an inwardly protruding portion that protrudes radially inwardly relative to the immediately adjacent portion of the tube-shaped member.

12. The catheter according to claim 7, wherein the tube-shaped member comprises: a tube having a distal end and a proximal end; and a distal end tip fixed to the distal end of the tube, the distalmost end portion of the tube-shaped member that includes the protruding portion being a part of the distal end tip.

13. The catheter according to claim 7, wherein the distalmost end portion of the tube-shaped member having the protruding portion has a radially outwardly facing outer peripheral surface and an axially facing distal end surface, the radially outwardly facing outer peripheral surface of the distalmost end portion of the tube-shaped member and the axially facing distal end surface of the distalmost end portion of the tube-shaped member intersecting one another at a rounded corner.

14. The catheter according to claim 7, wherein a distal portion of the tube-shaped member is curved.

15. The catheter according to claim 7, wherein the catheter satisfies a relational expression of
D1>D2,
d1<d2, and
T1>T2
D1 is an outer diameter of the distalmost end portion, d1 is an inner diameter of the distalmost end portion, T1 is a maximum thickness of the distalmost end portion, D2 is an outer diameter of the immediately adjacent portion of the tube-shaped member, d2 is an inner diameter of the immediately adjacent portion of the tube-shaped member, and T2 is a thickness of the immediately adjacent portion of the tube-shaped member.

16. A catheter having a hollow shape, the catheter comprising:
an outward protruding portion provided at a distalmost end portion and protruding outward in a radial direction; and
an inward protruding portion provided at the distalmost end portion and protruding inward in the radial direction.

17. The catheter according to claim 16, further comprising a distal end tip, wherein the outward protruding portion and the inward protruding portion are provided at the distal end tip.

18. The catheter according to claim 16, wherein a distal end portion of the outward protruding portion has a rounded shape.

19. The catheter according to claim 16, wherein a distal end portion of the inward protruding portion has a rounded shape.

20. The catheter according to claim 16, wherein the catheter satisfies a relational expression of
D1>D2,
d1<d2, and
T1>T2
D1 is a maximum outer diameter of the distalmost end portion, d1 is a minimum inner diameter of the distalmost end portion, T1 is a maximum thickness of the distalmost end portion, D2 is an outer diameter of a proximal portion provided on a proximal end side with respect to the distalmost end portion, d2 is an inner diameter of the proximal portion, and T2 is a thickness of the proximal portion.
